(19) United States
(12) Patent Application Publication
(10) Pub. No.: US 2003/0100935 A1
(43) Pub. Date: May 29, 2003

(54) Tanning device, especially full body tanning device

(76) Inventor: Walter Kratz, Eitorf (DE)

Correspondence Address: CROWELL & MORING LLP, INTELLECTUAL PROPERTY GROUP, P.O. BOX 14300, WASHINGTON, DC, 20044-4300, US

(21) Appl. No.: 10/312,338

(22) PCT Filed: Sep. 14, 2001
(86) PCT No.: PCT/EP01/10672

(30) Foreign Application Priority Data
Sep. 16, 2000 (DE) 200-16-020.6

(51) Int. Cl. A61N 5/06

(52) U.S. Cl. 607/90; 607/91

(57) ABSTRACT

The invention relates to a tanning apparatus, particularly a full body tanning apparatus which, for reducing the enery consumption and for improving a homogeneous tanning, has lamps embodied in a defined manner. The lamps are rod-like and are given an elongated-circular cross-section and are located in the tanning apparatus in such a way that the main axes are located roughly parallel to the lying surface or to a cover of the tanning apparatus in relation to the cross-section.

The invention relates to a tanning apparatus, particularly a whole-body tanning apparatus according to the preamble of claim 1.

In such tanning apparatuses lamps, particularly UV-lamps, which have a rod-like construction, are arranged in parallel in the longitudinal direction. The known, standardized lamps generally have a circular cross-section with a diameter of 26 or 38 mm.

DE 93 13 669.2 U1 discloses a tanning apparatus with a lower part and an upper part and optionally at least one side part for a whole-body tanning. Below a transparent lying surface of the lower part and a transparent cover of the upper part and the side part or parts are provided bar-shaped lamps and additional lamps, which can be connected in if required and which ensure an adequate tanning for different skin types. The additional lamps have a much smaller diameter than the main lamps and are located between the latter. The lamps and additional lamps are constructed as rod-like tubes with a circular cross-section.

To bring about a homogeneous tanning, said lamps and additional lamps having a circular cross-section are arranged as closely as possible on one another, which leads to a relatively large number of lamps in a tanning apparatus and to a corresponding power requirement.

The object of the invention is to create a tanning apparatus, particularly a whole-body tanning apparatus, which with a relatively small number of lamps ensures a uniform tanning.

According to the invention this object is achieved by the features of claim 1. Appropriate and advantageous developments appear in the subclaims and are described in the specific description.

An essential fundamental idea of the invention is for tanning apparatuses to move away from the known lamps having cross-sectionally circular glass tubes and to use lamps or fluorescent lamps having an elongated-circular cross-section and arrange them in such a way that the main axes are roughly parallel to a lying surface or cover of the tanning apparatus.

A glass tube for such lamps can have an oval cross-section, an elliptical cross-section or a flattened, circular cross-section. For example, in place of a round tube with a diameter of 38 mm, use can be made of an oval tube with a main axial length of approximately 50 mm and a secondary axial length of approximately 12 mm. Such an oval or glass tube with an elliptical cross-section then has main apices and secondary apices whereby a possibly light exit face is obtained if the latter cover the area between the two main apices.

In the case of a glass tube with an elongated-circular cross-section, it is also possible a lower and an upper side, as well as two arcuate portions to be formed, the lower and upper sides being parallel to one another.

The ratio of the main axial length to the secondary axial length can advantageously be between 1:0.25 and 1:0.6.

Per se known socket caps can be placed on the faces of rod-like lamps with an elongated-circular cross-section. It is also possible to provide adapted socket caps and also corresponding holding and contact receptacles on the apparatus side. For the known socket caps the rod-like lamps are terminally provided with a circular reception area.

In order to provide a possibly maximum light exit face, it is appropriate to apply longitudinally a reflecting coating to the inside or outside of the elliptical glass tube. It is alternatively possible to arrange a reflector device or the lamps can be arranged in reflectors having a complimentary construction.

It is advantageous if the reflecting layer or reflector device in each case extends from a main apex to the facing main apex. A light exit face of maximum extension is then formed.

The elliptical glass tubes of the lamps for the tanning apparatuses according to the invention, particularly whole-body tanning apparatuses, can also be used for fluorescent lamps having a rod, ring or U-shape and also other fields of use.

In the case of a tanning apparatus according to the invention, particularly a whole-body tanning apparatus, having at least a lower part and an upper part and optionally one or two side parts, the lamps are so placed below a transparent lying surface or a transparent cover of the upper part and optionally the side parts, that the main axis thereof is in each case roughly parallel to the transparent lying surface or the transparent cover. The main apices of the rod-like lamps with an elongated-circular cross-section are thus virtually adjacent to one another.

Appropriately a reflecting coating or also a reflector device is provided on the lamp side directed in opposition to the light exit face, the latter being directed towards the tanning area.

It can be advantageous to use lamps with an elongated-circular cross-section and lamps with a conventional circular cross-section in a tanning apparatus.

The essential advantages of tanning apparatuses, which are equipped with lamps having an elongated-circular, i.e. an oval, elliptical or flattened cross-section are that there is a larger light exit face and an optimized radiant flux with a smaller number of lamps. The arrangement of additional lamps having a smaller diameter between two lamps having a circular cross-section is unnecessary. In addition, the power costs are reduced.

The invention is described hereinafter relative to the highly diagrammatic drawings, wherein show:

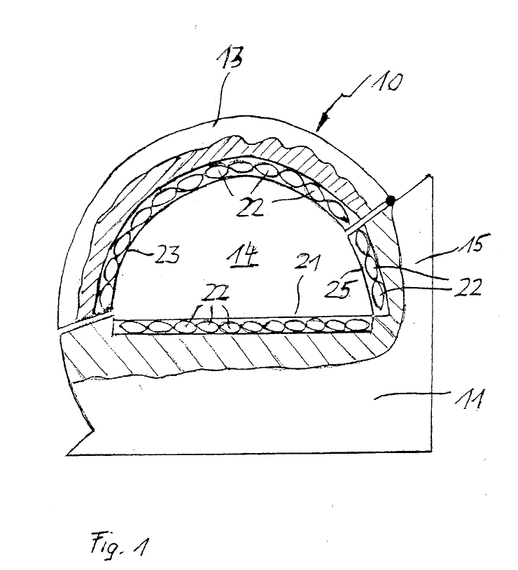

FIG. 1 A cross-section through a whole-body tanning apparatus according to the invention with lamps having an elongated-circular cross-section.

FIG. 2 A lamp having an oval cross-section.

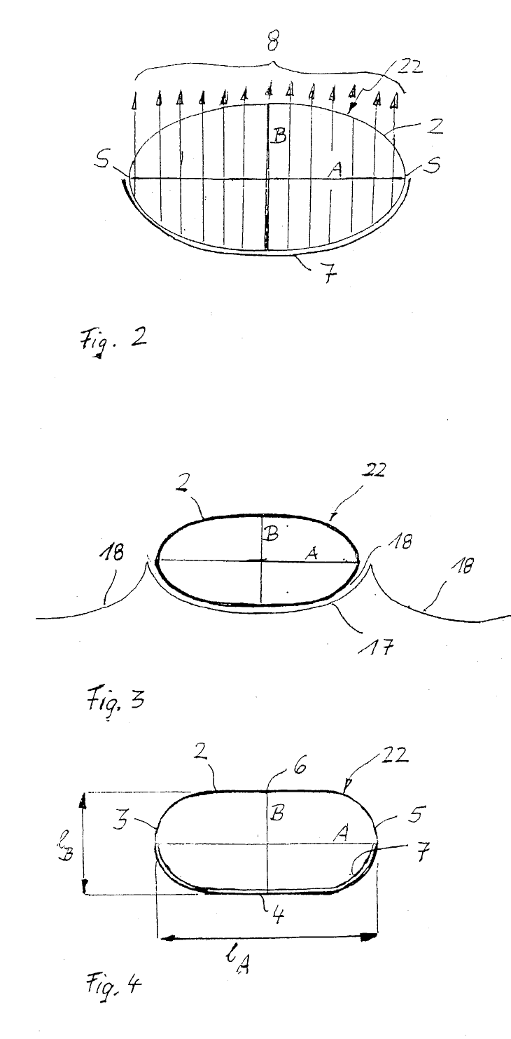

FIG. 3 A lamp having an elliptical cross-section.

FIG. 4 A lamp having a flattened cross-section.

The tanning apparatus 10 of FIG. 1 has a lower part 11 with a transparent lying surface 21 and below it lamps 22 as well as a side part 15 having a cover 25 and below it lamps 22. To the side part 15 is fixed in articulated manner an upper part 13 having a cover 23 and rod-like lamps 22 behind the latter. With the side part 15, the upper part 13 forms a cylindrical envelope surface and with the lower part 11 bounds a tanning area 14. In the installed state, the longitudinal axes of the lamps 22 are parallel to the longitudinal axis of the whole-body tanning apparatus 10 and the covers are transparent or transmitting for the UV radiation of the lamps 22 and can also have filters for undesired radiation.

In cross-section the lamps 22 are oval or elliptical and are arranged in such a way that the main axes A (cf. FIGS. 2 to 4) are parallel to the lying surface 21 or the covers 23 and 25.

The main apices S (cf. FIG. 2) of the lamps 22 are virtually adjacent to one another, and to the back is applied a reflecting coating 7, which can only be gathered from FIGS. 2 to 4 concerning alternative lamp constructions.

In the whole-body tanning apparatus 10 according to FIG. 1 a larger light exit face 8 is formed for each lamp 22 (cf. FIG. 2) and the orientation of the UV beams in the user plane or in the tanning area 14 is optimized. In addition, less lamps 22 are required per apparatus unit and an advantageous all-round tanning is brought about with a smaller number of lamps 22 and reduced power costs.

FIG. 2 shows in detail a rod-like or tubular lamp 22 with a glass tube 2 having an elongated-circular cross-section. The cross-section of the glass tube 2 is oval and has a main axis A and a secondary axis B. The lamp 22 is placed in the tanning apparatus 10 (FIG. 1) in such a way that the large light exit face 8 is directed towards the tanning area and can be fully utilized.

FIG. 2 shows that the light exit face 8 extends over the entire main axis A. At the back between the apices S formed by the main axis A is applied a reflecting coating 7 in the longitudinal direction of the. rod-like or tubular lamp 22. Fundamentally this reflecting coating 7 can be located on the inside or the outside of the glass tube 2.

It is also possible to provide a reflecting device in the tanning apparatus which should then have a complimentary construction to the elliptical lamp 22 or glass tube 2. The arrows in FIG. 2 indicate the light emission over the entire light exit face 8. It is clear that in the case of a lamp 22 with an elongated-circular, e.g. oval cross-section, the light exit face 8 is larger than with a conventional lamp having a circular cross-section and a comparable volume.

FIG. 3 shows a lamp with an elliptical glass tube 2. A comparison between FIGS. 2 and 3 reveals that the divergence between an oval and an elliptical cross-section is relatively small. In FIG. 3 a reflecting coating 7 is replaced by a reflecting device 17, which is shown in excerpt.

The reflecting device 17 has complimentary channels 18 for receiving a lamp 22 with an elongated-circular, elliptical or oval cross-section. A highly polished aluminium sheet is e.g. suitable as material for the reflecting device 17.

FIG. 4 shows an alternatively constructed lamp 22 in the area of a glass tube 2. The glass tube 2 has a cross-section, which can be looked upon as a flattened circle comprising a lower side 4 and an upper side 6, which are parallel to one another. The lower and upper sides 4, 6 are linked by arcuate portions 3, 5. The ratio of the main axial length l_{A} to the secondary axial length l_{B} of the glass tube 2 according to FIG. 3 is approximately 2:1. A reflecting coating 7 is applied to the inside.

1. Tanning apparatus, particularly whole-body tanning apparatus (10), which has at least a lower part (11) having a transparent lying surface (21) and lamps (22), as well as an upper part (13) having a transparent cover (23) and lamps (22), the lower part (11) and upper part (13) bounding a tanning area (14) and the lamps (22) have a tubular construction,
characterized in that
the lamps (22) have an elongated-circular cross-section with a main axis A and a secondary axis B and are arranged in such a way that the main axes A are roughly parallel to the transparent lying surface (21) or to the transparent cover (23).

2. Tanning apparatus according to claim 1,
characterized in that
the lamps (22) have glass tubes (2) with an oval cross-section.

3. Tanning apparatus according to claim 1,
characterized in that
the lamps (22) have glass tubes (2) with an elliptical cross-section.

4. Tanning apparatus according to claim 1,
characterized in that
the lamps (22) have glass tubes (2) with a cross-section representing a flattened circle.

5. Tanning apparatus according to claim 4,
characterized in that
the glass tube (2) has a lower side (4) and an upper side (6), as well as two arcuate portions (3, 5), the lower and upper sides (4, 6) are parallel to one another and are linked by the arcuate portions (3, 5).

6. Tanning apparatus according to one of the preceding claims,
characterized in that
the lamps (22) with an elongated-circular cross-section have a main axis A and a secondary axis B and that the ratio of the main axial length l_{A} to the secondary axial length l_{B} is 1:0.25 to 1:0.6.

7. Tanning apparatus according to one of the preceding claims,
characterized in that
the glass tubes (2) of the lamps (22) are frontally provided with reception areas for conventional socket caps.

8. Tanning apparatus according to one of the preceding claims,
characterized in that
the lamps (22) are provided with a reflecting coating (7) and that the reflecting coating (7) is in each case back-applied to an inside or outside of the glass tube (2), accompanied by the formation of a light exit face (8).

9. Tanning apparatus according to claim 8,
characterized in that
the lamps (22) are arranged in such a way that the reflecting coating (7) or a reflecting device (18) is at the back and the light exit faces (8) of the lamps (22) are directed towards the tanning area (14).

10. Tanning apparatus according to one of the preceding claims,
characterized in that
lamps (22) with an elongated-circular cross-section and lamps with a circular cross-section are arranged.
